(19) United States
(12) Patent Application Publication
(10) Pub. No.: US 2008/0200871 A1
(43) Pub. Date: Aug. 21, 2008

(54) Valve System For a Medical Device Having an Inflatable Member

(76) Inventors: Charles R. Slater, Fort Lauderdale, FL (US); Brett E. Naglreiter, Austin, TX (US); Peter K. Kratsch, Davie, FL (US); Alejandro Espinosa, Miami, FL (US); Scott T. Smith, Stuart, FL (US); Scott L. Jahrmarkt, Miami Beach, FL (US)

Correspondence Address: GORDON & JACOBSON, P.C., 60 LONG RIDGE ROAD, SUITE 407, STAMFORD, CT, 06902, US

(21) Appl. No.: 11/813,164

(22) PCT Filed: Jan. 13, 2006
(86) PCT No.: PCT/US2006/001458

§ 371 Date: Nov. 21, 2007

Related U.S. Application Data
(60) Provisional application No. 60/644,265, filed on Jan. 14, 2005.

(51) Int. Cl. A61M 29/00

(52) U.S. Cl. 604/96.01; 606/194

(57) ABSTRACT

Methods and apparatus for treating the interior of a blood vessel include a variety of improved catheter designs, methods and apparatus for accessing and occluding a blood vessel.

### BACKGROUND OF THE INVENTION

1. Field of the Invention

The invention relates to the treatment and correction of venous insufficiency. More particularly the invention relates to a minimally invasive procedure using a catheter-based system to treat the interior of a blood vessel. The invention has particular application to varicose veins although it is not limited thereto.

2. Background

The human venous system of the lower limbs consists essentially of the superficial venous system and the deep venous system with perforating veins connecting the two systems. The superficial system includes the long or great saphenous vein and the short saphenous vein. The deep venous system includes the anterior and posterior tibial veins which unite to form the popliteal vein, which in turn becomes the femoral vein when joined by the short saphenous vein.

The venous systems contain numerous one-way valves for directing blood flow back to the heart. Venous valves are usually bicuspid valves, with each cusp forming a sac or reservoir for blood which, under pressure, forces the free surfaces of the cusps together to prevent retrograde flow of the blood and allow antegrade flow to the heart. An incompetent valve is a valve which is unable to close because the cusps do not form a proper seal and retrograde flow of blood cannot be stopped.

Incompetence in the venous system can result from vein dilation. Separation of the cusps of the venous valve at the commissure may occur as a result. Two venous diseases which often involve vein dilation are varicose veins and chronic venous insufficiency.

The varicose vein condition includes dilatation and tortuosity of the superficial veins of the lower limb, resulting in unsightly discoloration, pain and ulceration. Varicose veins often involve incompetence of one or more venous valves, which allow reflux of blood from the deep venous system to the superficial venous system or reflux within the superficial system.

Varicose veins are compatible with long life and rarely cause fatal complications, but the condition significantly decreases the quality of life. Patients complain primarily of leg fatigue, dull, aching pains, ankle swelling and ulcerations. Occasionally, thrombosis occurs in dilated subcutaneous channels, resulting in local pain, induration, edema, inflammation, and disability. In addition to those problems, the high visibility of the unattractive rope-like swellings and reddish skin blotches causes considerable distress for both men and women. Lastly, varicose eczema, which is a local reddened swollen and itching skin condition can occur and can spread to distant parts of the body (called an “Id reaction”).

Phlebosclerosis, the destruction of venous channels by the injection of sclerosing agents, has been used to treat varicose veins since 1853, when Cassaignae and Ebout used ferric chloride. Sodium salicylate, quinine, urea, and sodium chloride have also been used, but the agent more recently favored is sodium tetradecyl sulfate. In order for phlebosclerosis to be effective, it is necessary to evenly dispense the sclerosing agent throughout the wall of the vein without using toxic levels of the sclerosing agent. This is not particularly difficult for the smaller veins. However, it is quite difficult or nearly impossible in larger veins. When a larger vein is injected with a sclerosing agent, the sclerosing agent is quickly diluted by the substantially larger volume of blood which is not present in smaller veins. The result is that the vein is sclerosed (injured) only in the vicinity of the injection. If the procedure is continued, and the injections are far apart, the vein often assumes a configuration resembling sausage links. The problem cannot be cured by injecting a more potent solution of sclerosing agent, because the sclerosing agent may become toxic at such a concentration.

U.S. Pat. No. 5,676,962 discloses an injectable micro foam containing a sclerosing agent. The microfoam is injected into a vein where it expands and, theoretically, achieves the same results as a larger quantity of sclerosing agent without the toxicity. Such foam is presently manufactured under the trademark Varisolve® by Provensis, Ltd., London, England. Recent clinical trials of the foam indicate a success rate of 81%.

Until recently, the preferred procedure for treating the great saphenous vein was surgical stripping. This highly invasive procedure involves making a 2.5 cm incision in the groin to expose the saphenofemoral junction, where the great saphenous vein and its branches are doubly ligated en masse with a heavy ligature. The distal portion of the vein is exposed through a 1-cm incision anterior to the medial malleolus, and a flat metal or plastic stripper is introduced to exit in the proximal saphenous vein. The leg is held vertically for 30 seconds to empty the venous tree before stripping the vein from the ankle to the groin. If the small saphenous vein is also incompetent, it is stripped at the same time from an incision posterior to the lateral malleolus to the popliteal space. After stripping the veins, the leg is held in the vertical position for three to four minutes to permit broken vessel ends to retract, constrict, and clot.

After the stripping procedure, collateral veins are removed by the avulsion-extraction technique. By working through small (5 to 8 mm) transverse incisions, segments of vein 10 to 20 cm long can be removed by dissecting subcutaneously along the vein with a hemostat, and then grasping, avulsing, and removing the vein. With practice, long segments of vein in all quadrants can be removed through these small incisions. No attempt is made to ligate the branches or ends of the veins, since stripping has shown it to be unnecessary. Bleeding is controlled by elevation and pressure for two to four minutes. As many as 40 incisions are made in severe cases, but their small size and transverse direction permit closure with a single suture.

Before closure of the incisions, a rolled towel is rolled repeatedly from the knee to the ankle and from the knee to the groin to express any clots that may have accumulated. The groin incision is approximated with three 5-0 nylon mattress sutures and all other incisions are closed with a single suture.

As can be readily appreciated, the stripping and avulsion-extraction procedures are relatively invasive and require significant anesthesia. It can therefore be appreciated that it would be desirable to provide an alternative, less invasive procedure which would accomplish the same results as stripping and avulsion-extraction.

Recently, a number of patents have issued disclosing the treatment of varicose veins with RF energy. Illustrative of these recent patents are: U.S. Pat. No. 6,200,312 entitled “Expandable Vein Ligator Catheter Having Multiple Electrode Leads”; U.S. Pat. No. 6,179,832 entitled “Expandable Catheter Having Two Sets of Electrodes”; U.S. Pat. No. 6,165,172 entitled “Expandable Vein Ligator Catheter and Method of Use”; U.S. Pat. No. 6,152,899 entitled “Expandable Catheter Having Improved Electrode Design, and Method for Applying Energy”; U.S. Pat. No. 6,071,277 entitled “Method and Apparatus for Reducing the Size of a Hollow Anatomical Structure”; U.S. Pat. No. 6,036,687 entitled “Method and Apparatus for Treating Venous Insufficiency”; U.S. Pat. No. 6,033,398 entitled “Method and Apparatus for Treating Venous Insufficiency Using Directionally Applied Energy”; U.S. Pat. No. 6,014,589 entitled “Catheter Having Expandable Electrodes and Adjustable Stent”; U.S. Pat. No. 5,810,847 entitled “Method and Apparatus for Minimally Invasive Treatment of Chronic Venous Insufficiency”; U.S. Pat. No. 5,730,136 entitled “Venous Pump Efficiency Test System And Method”; and U.S. Pat. No. 5,609,598 entitled “Method and Apparatus for Minimally Invasive Treatment of Chronic Venous Insufficiency”. These patents generally disclose a catheter having an electrode tip which is switchably coupled to a source of RF energy. The catheter is positioned within the vein to be treated, and the electrodes on the catheter are moved toward one side of the vein. RF energy is applied to cause localized heating and corresponding shrinkage of the adjacent venous tissue. After treating one section of the vein, the catheter can be repositioned to place the electrodes to treat different sections of the vein.

Although this procedure has gained acceptance and is less invasive than the stripping and avulsion-extraction procedures, there are several disadvantages to it. In particular, RF treatment is actually quite slow and painful and the patient must be sufficiently anaesthetized along the entire length of the veins to be treated. In addition, repositioning the catheter is time consuming thus requiring anesthesia for a prolonged period. Moreover, the RF treatment is incomplete, as only a portion of the vein wall is actually treated, i.e. the portion contacting the electrode. The partially treated vein may eventually recanalize. Furthermore, tributary veins remain unaffected and must be treated separately. In addition, for even and consistent cauterization, RF treatment requires that the practitioner be keenly aware of the procedure time. If RF energy is applied for too long, it can cause undesired burns. If RF energy is not applied long enough, the treatment is ineffective.

In addition to RF treatment, laser treatment has been used with some success. Laser treatment shares many of the disadvantages of RF treatment. In particular, as with the RF devices, the practitioner must be very careful as to the intensity and duration of the treatment to assure that the treatment is effective but without causing undesired burns.

Parent application Ser. No. 09/898,867 discloses an apparatus for delivering an intravascular drug such as a sclerosing agent (or a microfoam sclerosing agent) to a varicose vein. The apparatus includes a catheter having three concentric tubes. The innermost tube has a guide wire lumen and an inflation lumen. The distal end of the innermost tube has an integral inflatable occlusion balloon in fluid communication with the inflation lumen. The intermediate tube has a lumen through which the innermost tube extends. The distal end of the intermediate tube has a self-expanding balloon with a plurality of fluid pores in fluid communication with the intermediate tube lumen. The outer tube has a lumen through which the intermediate tube extends. Sclerosing agent is dispensed through the intermediate tube to pores located at the distal end of the intermediate tube or in the self-expanding balloon. Veins are sclerosed as the self-expanding balloon is pulled through and ultimately out of the vein.

While particular methods and apparatus were disclosed in the parent application for occluding the blood vessel, dispensing sclerosing agent, and locating tributaries, it will be appreciated that it would be desirable to have additional manners of accomplishing the same.

### BRIEF DESCRIPTION OF THE DRAWINGS

The structure and method of using the invention will be better understood with the following detailed description of embodiments of the invention, along with the accompanying illustrations, in which:

FIGS. 1A depicts one embodiment of the invention comprising a valve assembly. FIGS. 1B and 1C are cross-sectional views of the valve assembly in FIG. 1A;

FIG. 2 shows a shaded, partially-transparent view of the valve assembly in FIGS. 1A through 1C;

FIG. 3 shows an exploded view of the components of the valve assembly in FIGS. 1A through 1C;

FIG. 4 is a shaded, partially transparent assembly view depicting the housing assembly at the proximal end of the catheter system;

FIG. 5 illustrates one embodiment of the proximal end of the catheter and the housing assembly;

FIG. 6 is an exploded view of the embodiment depicted in FIG. 5;

FIGS. 7A through 7C are cross sectional views of one embodiment of the distal end construction of the device;

FIGS. 8A and 8B show an inflatable bladder in an open and closed positions.

### DETAILED DESCRIPTION OF THE PREFERRED EMBODIMENT

Previous patent applications by the inventors, Ser. Nos. 10/922,221 and 10/922,123 both filed on Aug. 19, 2004 and herein incorporated by reference in their entirety, have disclosed catheter systems for infusion of fluids into anatomical spaces and lumens. This invention disclosure teaches several improvements to these and other catheter systems which may be utilized in part or all together to improve the manufacture and use of catheter systems. These improvements may be adapted to any catheter system comprising an inflatable assembly, including but not limited to angioplasty catheters, stent delivery catheters, catheters for delivering implantable medical devices and others.

### A. Improved System for Inflating and Deflating a Bladder Tube or Other Balloon

In order to operate the catheter system as previously described, it is necessary to introduce fluid pressure into inflatable elements, maintain that pressure for a period of time, and then release the pressure. In the case of a bladder tube used to block flow through a catheter, an inflatable tubular balloon disposed about or within a lumen of the catheter is filled with fluid by means of a syringe or fluid pump until the bladder contacts the lumen wall, blocking flow through the lumen. A fluid may be a foam, a liquid, a gaseous substance, a suspension or any other flowable substance. The previously described method required the user to: open a valve (stopcock); depress a fluid-filled syringe until blockage of the lumen was assessed by some means; close the valve; and then, when desired, detach the syringe and opening the valve to deflate the balloon.

1. Valve Assembly

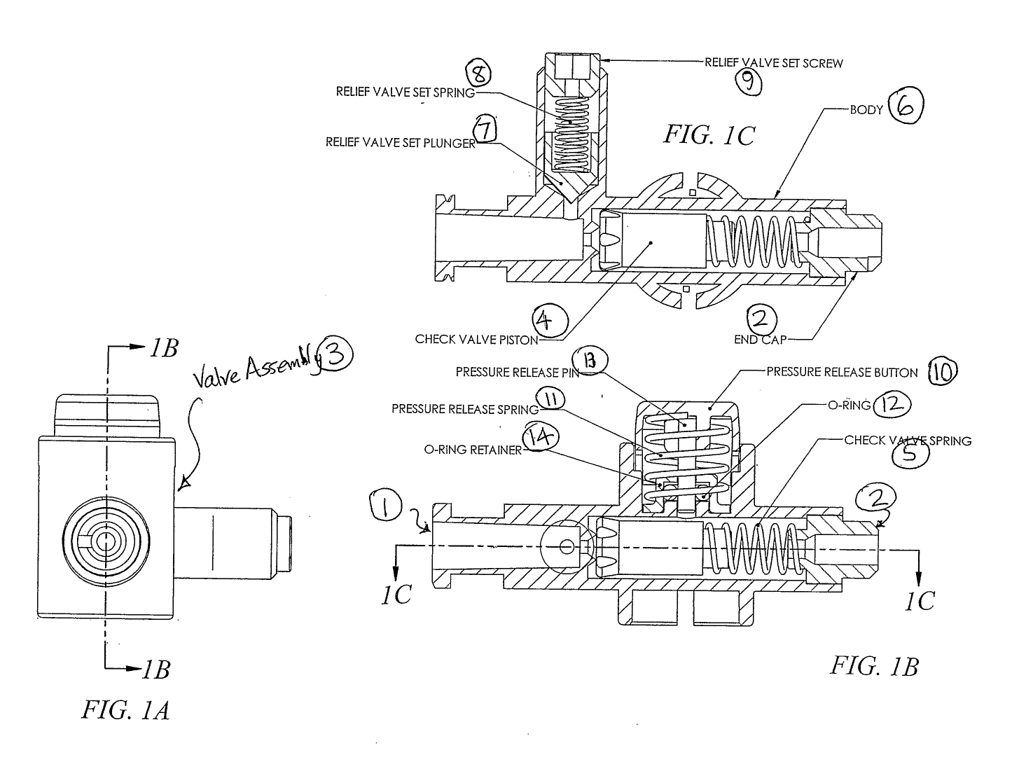

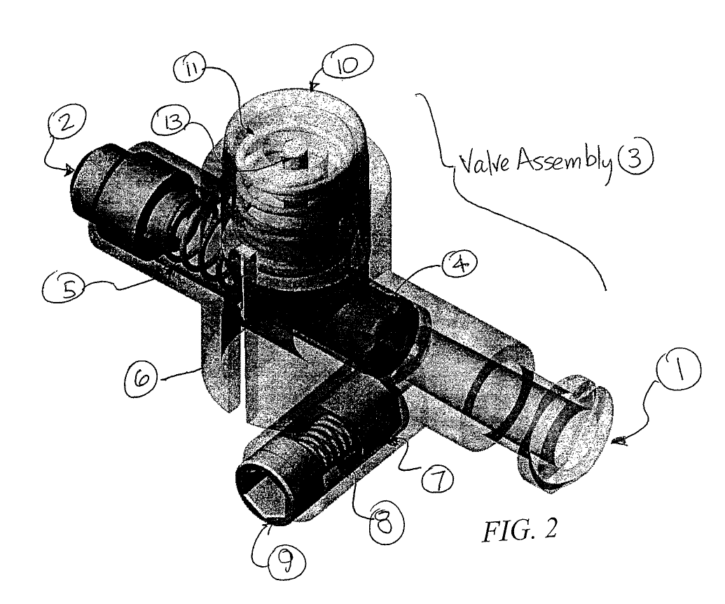

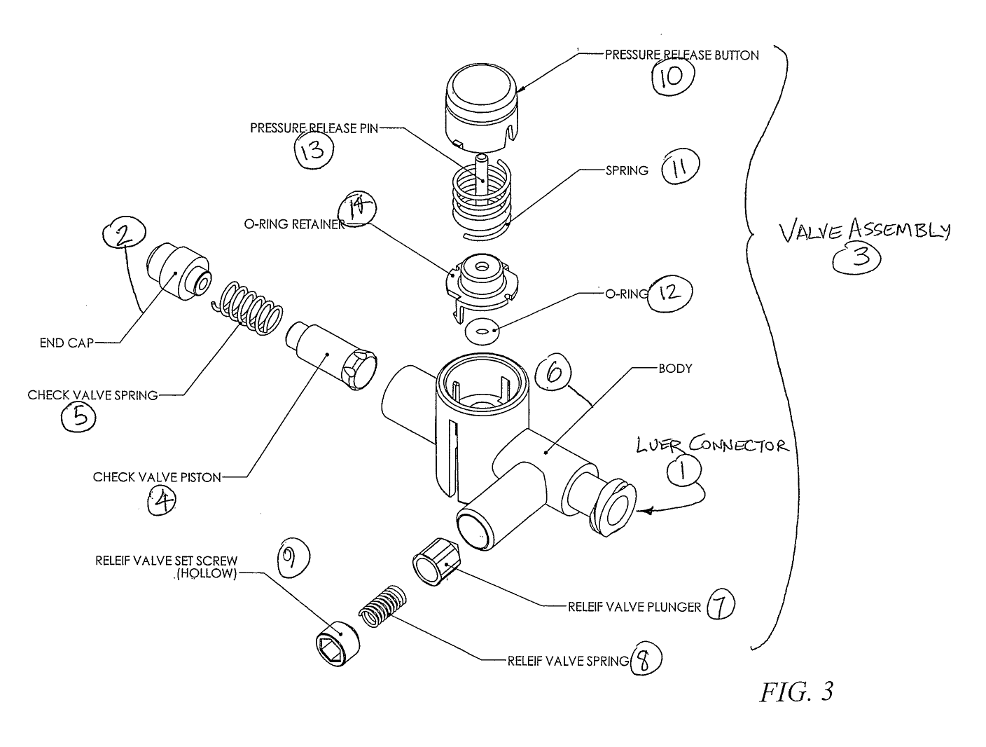

The present invention is an integrated valve assembly which greatly simplifies the inflation, holding, and deflation of an inflatable member such as a balloon. FIGS. 1A through 1C depict one embodiment of the invention comprising a Bladder Release Valve Assembly 3; FIG. 2 shows a shaded, partially-transparent view of the Bladder Release Valve Assembly 3; and, FIG. 3 shows an exploded view of the components of the Bladder Release Valve Assembly 3.

Referring to FIGS. 1A through 1C, the Bladder Release Valve Assembly 3 comprises a Body 6 in which two valve assemblies are implemented. The inflatable bladder tube (not shown) is connected to End Cap 2.

2. Check Valve

A check valve comprising a Check Valve Piston 4 and a Check Valve Spring 5 allows fluid to pass freely from the Body 6 of the Valve Assembly 3 into the bladder tube.

In one embodiment for operating the device, the user attaches a fluid-filled syringe (for example, a 1-cc syringe filled with water), not shown, to Luer Connector 1. The user then depresses the syringe fully to inflate the bladder tube through the check valve. As fluid pressure rises, fluid flows into the bladder tube, inflating it.

3. Pressure-Relief Valve

In one embodiment, a pressure-relief valve is provided, comprising a Relief Valve Plunger 7, a Relief Valve Spring 8, and a Relief Valve Set Screw 9. As the user depresses the plunger of the syringe beyond the amount needed to fully inflate the bladder tube, any excess fluid flow then causes the pressure-relief valve to open, venting the excess fluid through the hollow Relief Valve Set Screw 9. Therefore, the user has only to depress the syringe fully; fluid exiting the hollow Relief Valve Set Screw 9 will indicate to the user that the bladder tube is fully inflated, blocking the catheter lumen.

4. Deflation Valve Apparatus

The Check Valve Piston 4 is designed to fit closely within proximal end of the cylindrical bore of the Body 6 where the Piston 4 seals against a ridge formed in the Body 6. However, the distal portion of the Piston 4 is of a reduced diameter, so that it can be cocked within the cylindrical bore of the Body 6. In one embodiment, the deflation apparatus comprises a Pressure Release Button 10, a Spring 11, an O-ring 12, a Pressure Release Pin 13, and an O-ring Retainer 14. When the Pressure Release Button 10 is depressed against the force of the Spring 11, the Pressure Release Pin 13 is driven through the O-ring 12, directing force against the side of the Check Valve Piston 4; when the Check Valve Piston 4 is cocked sideways, the seal formed between its left end and the ridge within the cylindrical bore of the Body 6 is broken, allowing fluid to flow retrograde through the check valve toward the Luer Connector 1. The fluid then exits the Luer Connector 1 by pushing the syringe piston backward, because the user is no longer applying depressing force against it; if the syringe has been disconnected from the Luer Connector 1, then the fluid simply exits from the Luer Connector 1. In any event, as fluid flows back through the check valve, the bladder tubing is deflated, restoring fluid flow capability to the lumen in which the bladder tubing resides.

While the aforementioned Bladder Release Valve Assembly 3 is described for use with a bladder tube valve, it will be obvious that the same invention can be used to inflate, hold, and deflate any inflatable structure that is amenable to inflation to a constant pressure.

In one example, the balloon used in the previously described catheter system to occlude body lumens may be controlled in a similar manner. Doing so would result in an easily controlled method of occluding body lumens such as the greater saphenous vein. Rather than the previously described method in which the user must determine by other means when the balloon is inflated sufficiently to occlude the vein, using the present invention the user simply has to depress a syringe plunger and watch for fluid to exit the relief valve opening. For such an application, a balloon would be used which utilizes substantially less pressure to inflate than the pressure that would damage the vein, and the pressure relief valve would be calibrated (using Relief Valve Set Screw 9) for a pressure above the balloon-inflation pressure but less than the vein-injury pressure. Similarly, such a device may also be configured for occluding other body lumens such as arteries, lymphatic ducts, the gastrointestinal tract and genitourinary passages.

B. Housing Assembly with Tri Hub

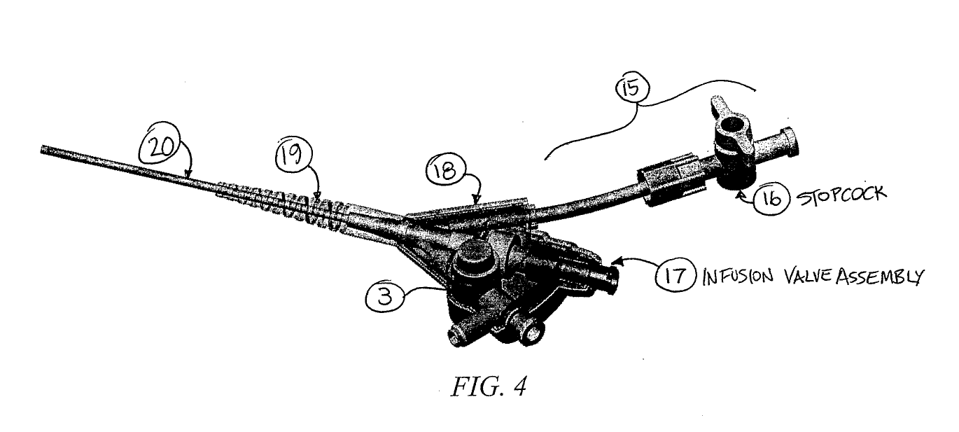

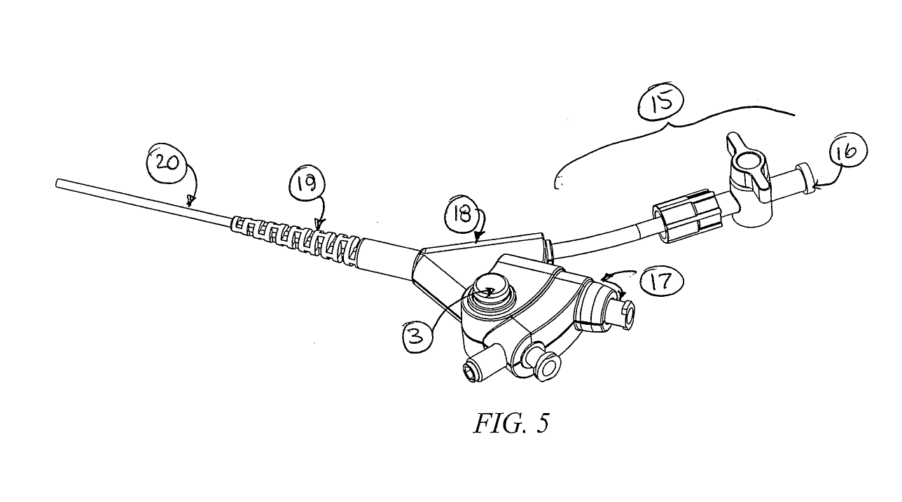

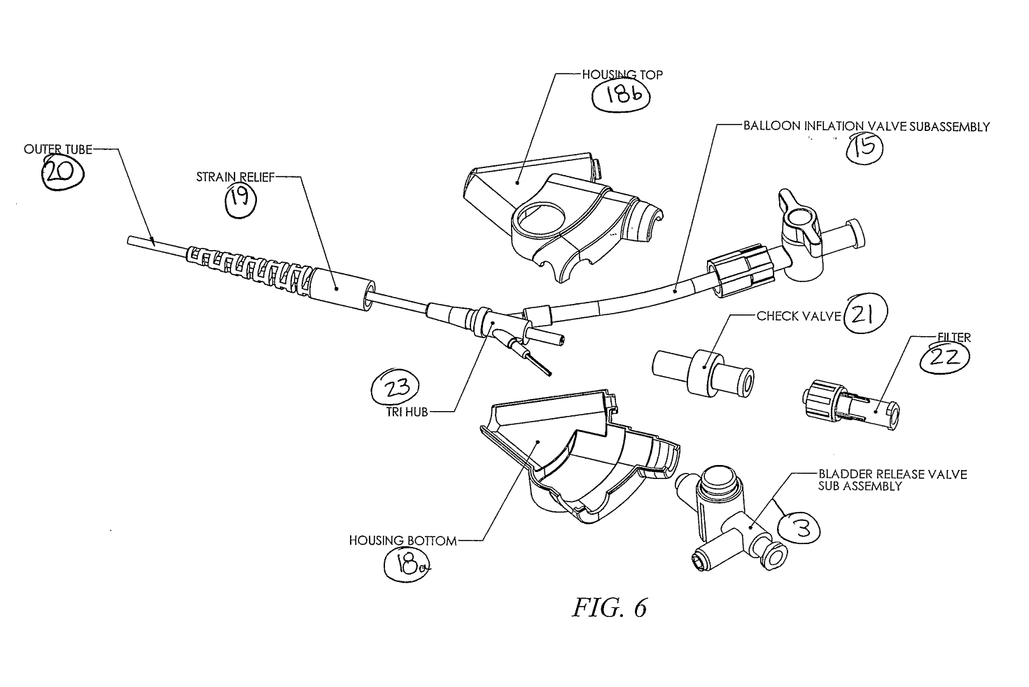

FIG. 4, a shaded, partially transparent assembly drawing, depicts the housing assembly at the proximal end of the catheter system. The proximal end of the catheter's Outer Tube 20 is joined to the Housing 18 by means of a Strain Relief 19 to prevent kinking where the flexible catheter joins the rigid housing assembly. FIG. 5 is a line drawing of the proximal end of the catheter and the housing assembly. FIG. 6 is an exploded view of this same area.

Referring to FIG. 6, the housing assembly comprises a Housing Top 18b, a Housing Bottom 18a, the catheter Outer Tube 20, the Strain Relief 19, a Balloon Inflation Valve Subassembly 15, a Check Valve 21, a Filter 22, a Tri Hub 23, and the Bladder Release Valve Assembly 3, previously described.

1. Housing

In one embodiment, the Housing 18 serves to contain and mount the Bladder Release Valve Assembly 3, Infusion Valve Assembly 17 and the Tri Hub 23. Attached to Housing 18 are the Balloon Inflation Valve Sub-assembly 15 (which contains the Stopcock 16) and the Strain Relief 19, which covers the Outer Tube 20.

The catheter attached to the housing assembly may have one or more lumens. In a preferred embodiment, the catheter attached to the housing assembly contains three lumens, either as discrete lumens or as tubes within a tube, or as a combination of tubes and lumens. One lumen is used to supply infusate fluid to the catheter for delivery to a selected body region. A second lumen is provided to allow inflation and deflation of an occlusion balloon used to block flow within a body lumen. The third lumen is used to inflate and deflate the bladder tubing, which operates as an internal valve mechanism within the catheter to control the egress and ingress of fluid from and to the catheter.

2. Bladder Release Valve Sub-Assembly

The Bladder Release Valve Assembly 3 has been previously described in detail and acts to control the bladder tubing and is the point to which a syringe is attached for controlling the bladder tubing.

3. Infusion Valve Assembly

The Infusion Valve Assembly 17 includes an optional check valve and optional filter; it constitutes the connection to which a syringe or other means is attached to provide fluid under pressure for delivery to the selected body region. The Balloon Inflation Valve Sub-assembly 15 contains a Stopcock 16 to control inflation of the occlusion balloon and constitutes the connection to which a syringe is attached to inflate the occlusion balloon at the distal end of the catheter.

4. Balloon Inflation Valve Sub-Assembly

The Balloon Inflation Valve Sub-assembly 15 comprises a length of flexible tubing and the Stopcock 16. A syringe or other pressurized-fluid means is attached to the open end of the Stopcock 16. To inflate the occlusion balloon, the Stopcock 16 is moved to the open position and the syringe is depressed to supply pressurized fluid to inflate the occlusion balloon. When the occlusion balloon has been inflated to the desired state, the Stopcock 16 is moved to the closed position to maintain the occlusion balloon inflation. When it is desired to deflate the occlusion balloon, the syringe is detached and the Stopcock 16 is moved to the open position.

5. Infusion Valve Assembly

The Infusion Valve Assembly 17 comprises the Check Valve 21 and the Filter 22. A syringe or other source of pressurized fluid (liquid, gas, or foam) is attached to the open end of the Filter 22. When the catheter has been positioned and made ready for infusion, depressing the syringe attached to the Infusion Valve Assembly 17 causes fluid to pass through the Filter 22, the Check Valve 21, then by means of the Tri Hub 23 into the infusion lumen of the catheter, and thence to the desired body region. The purpose of the Check Valve 21 is to reduce the tendency of blood to enter the catheter before pressurized infusate fluid is supplied, with the possible consequence of clogging the infusion holes. The Check Valve 21 also assists in maintaining the primed infusion lumen during prep. The purpose of the Filter 22 is to minimize or eliminate particulate contamination of the infusate fluid that could result in clogging of the infusion holes or harm to the patient by embolization. Either the Filter 22 or the Check Valve 21, or both, are required in the catheter system. Also, in other embodiments, the Filter 21 and the Check Valve 22 may be assembled in a different configuration or reverse order.

### C. Distal End Construction

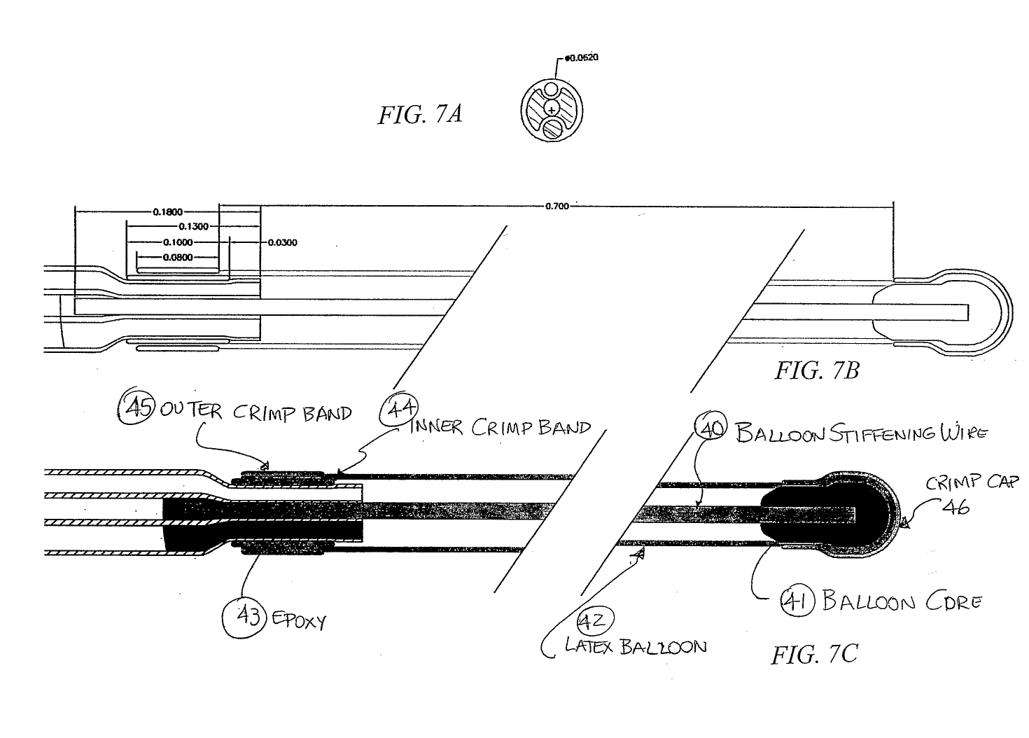

The distal end construction of the device is shown in FIG. 7.

1. Fixation of Balloon Core Onto the Stiffening Wire

A metal Balloon Core 41 is crimped onto a Balloon Stiffening Wire 40. The Balloon Core 41 may have an enlarged distal most diameter, as shown in FIG. 7, to aid in retention of a Crimp Cap 46.

2. Balloon Stiffening Wire Encapsulation

The Balloon Stiffening Wire 40, made of an elastic or superelastic metal (e.g., nitinol), is inserted into the distal end of the catheter within the central (infusate) lumen and is bonded in place by wicking a suitable adhesive (e.g., UV-cure epoxy) into the end of the catheter lumen; the encapsulation is shown as Epoxy 43 in FIG. 7. Once cured, the Epoxy 43 anchors the Balloon Stiffening Wire 40 in place and prevents leakage of the infusate into a Latex Balloon 42. The superelastic Balloon Stiffening Wire 40 allows for the balloon subassembly to be extremely flexible and robust, which can aid in vessel trackability and reduces vessel trauma. Additionally, the Balloon Stiffening Wire 40 offers a smooth transition of flexibility from the body of the catheter to its distal end.

3. Fixation of the Latex Balloon Onto the Catheter

In one embodiment, the Latex Balloon 42 may be affixed to the distal end of the catheter via crimp bands. An Inner Crimp Band 44 is compressed onto the distal end of the catheter tube, which has been previously reduced in diameter by some means such as pulling or pushing the tubing through a heated reducing die. The open end of the closed-end Latex Balloon 42 is placed over the Balloon Core 41 and the Balloon Stiffening Wire 40 until it extends over the Inner Crimp Band 44, and an Outer Crimp Band 45 is then compressed onto the assembly over the Inner Crimp Band 44 and the open end of the Latex Balloon 42, trapping the balloon material between the Inner Crimp Band 44 and the Outer Crimp Band 45.

The inflation range of the balloon is larger than expected because the Latex Balloon 42 is assembled “loose” on the assembly, but the ends are stretched to a smaller diameter before crimping to prevent any “folding” between the Crimp Bands 44, 45. This design also allows for the looseness of the balloon to be uniformly distributed around the assembly, allowing the balloon to inflate concentrically.

4. Fixation of Crimp Cap Onto Balloon and Balloon Core

The Crimp Cap 46 is place over the closed end of the Latex Balloon 42, which contains the Balloon Core 41. The Crimp Cap 46 is made of a suitable ductile metal (e.g., stainless steel), which covers the closed end of the Latex Balloon 42 and a substantial portion of the Balloon Core 41, including the possibly enlarged distal portion of the Balloon Core 41. The Crimp Cap 46 is then crimped so that it compresses the balloon material against the Balloon Core 41, trapping the Balloon Core 41 and the Balloon Stiffening Wire 40 in place within the Latex Balloon 42.

The Crimp Cap 46 may alternately have an extended portion distal to its hollow portion; the extended portion may contain additional features, such as a monorail guidewire lumen.

### D. Distal Monorail Assembly

The catheter designs taught in the inventors' previous applications describe an apparatus and method for introducing a catheter to a body lumen for the purpose of infusing a fluid. The methods previously taught describe introducing the catheter into a vessel so that it follows the vessel passively until the distal end of the catheter is positioned where the user desires.

An alternative means of introducing catheters, used commonly by radiologists and cardiologists, is to first introduce a flexible guidewire. Once the guidewire has been advanced inside the vessel beyond the place where the catheter tip is desired to go, the catheter is placed onto the guidewire. Once engaged over the guidewire, the catheter is pushed distally so that it tracks along the guidewire until its distal tip reaches the desired position. Simple catheters consist of hollow tubes through which the guidewire can travel; hence, it is easy to pass a simple tubular catheter over a guidewire. However, multi-lumen catheters such as the present catheter system often do not contain a hollow lumen extending from the proximal end of the catheter to the distal end, but rather contain a short tubular lumen at some location near the distal end of the catheter so that the guidewire enters and exits that lumen substantially near the distal end of the catheter.

Such an arrangement, usually called a “monorail,” allows facile placement of the catheter into engagement of the guidewire without requiring another lumen running the full length of the catheter, and such an arrangement allows the use of relatively short guidewires. (If a simple, through-lumen catheter is to be introduced along a guidewire, the length of the guidewire outside the body must be greater than the length of the catheter, so that the catheter can be fully positioned onto the guidewire while the guidewire is held near where it enters the body; then, before letting go of the guidewire at the body-entry point, the most proximal end of the guidewire may be grasped to allow pushing the catheter along the guidewire into the body lumen. In this manner, the guidewire is never allowed to “float” without being held by the user.)

In order to effect a monorail configuration of the present catheter system, it may be desirable to provide an extended atraumatic tip longer than the atraumatic tip at the distal end of the occlusion balloon described in the inventors' previous patent applications. The new, extended atraumatic tip can contain a separate tubular lumen which extends from the very distal tip of the atraumatic tip (hence, the very distal tip of the catheter) a short distance proximally, and which exits just distal to the occlusion balloon. In this manner, there exists a short lumen contained solely within the extended atraumatic tip through which the guidewire might be passed.

In use, once the guidewire has been placed in the body, the proximal end of the guidewire is passed into the distal guidewire opening of the catheter's atraumatic tip. As the catheter is advanced a short distance along the guidewire, the proximal end of the guidewire exits the side opening just distal of the occlusion balloon. Once the proximal end of the guidewire has exited the proximal end of the guidewire lumen, the guidewire end may be grasped, and the catheter pushed along the guidewire so that it enters the body and follows the guidewire to the desired position within the body. The user could also advance the guide wire and the catheter simultaneously using a “push/pull” technique. In addition, the catheter will be smaller because it does not include a separate lumen for a guidewire, and it will be compatible with a smaller French size introducer because of the guidewire residing outside the catheter bore.

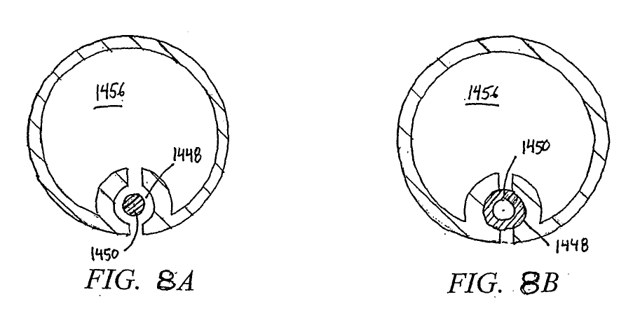

FIGS. 8A and 8B depict an inflatable bladder and infusion tube assembly which can be used with the valves and balloon of the invention. It comprises a hollow flow regulating tube 1450, having a central lumen 1452 is positioned within the side lumen 1448. The tube 1450 has an open proximal end and a closed distal end. The proximal end may be provided with a releasable connector such as a luer fitting for connection to a source of inflation media. Alternatively, the central lumen may be in direct communication with a variable volume chamber in the proximal manifold or hand piece for the catheter.

The outside diameter of the flow regulating tube 1450 is moveable from a first, reduced diameter to a second enlarged diameter upon introduction of inflation media into the central lumen 1452. The outside diameter of the tube 1450 in the first, relaxed configuration is less than the inside diameter of the lumen within which it resides, such as side lumen 1448. In this configuration, a medicament or other agent in the infusion lumen 1456 is capable of flowing past or around the hollow tube 1450 to exit out of the elution hole 1454. See FIG. 8A. Introduction of inflation media into central lumen 1452 causes an enlargement of the outside diameter of the tube 1450 such that it occludes the flow path between the infusion lumen 1456 and the exterior of the catheter body. See FIG. 58B. Further information regarding this arrangement can be found in Published U.S. Patent Application 20050113798 A1.

While this invention has been particularly shown and described with references to embodiments thereof, it will be understood by those skilled in the art that various changes in form and details may be made therein without departing from the scope of the invention. For all of the embodiments described above, the steps of the methods need not be performed sequentially.

1. A valve assembly for use with an inflatable medical device, comprising:
a valve body having a fluid inlet and a fluid outlet;
a first valve assembly located between said inlet and said outlet, said first valve assembly permitting fluid flow from said inlet to said outlet and preventing fluid flow from said outlet to said inlet; and
a second valve assembly arranged to automatically release fluid from said valve body before passing through said outlet when a preset fluid pressure is exceeded.

2. A valve assembly according to claim 1, further comprising: a third valve assembly being operable to allow fluid from said outlet to exit said valve body.

3. A valve assembly according to claim 1, wherein:
said second valve assembly is located between said first valve assembly and said inlet.

4. A valve assembly according to claim 2, wherein:
said first valve assembly has a movable valve seal, and said third valve assembly is located between said seal and said outlet.

5. A valve assembly according to claim 1, wherein:
said inlet is provided with a Luer connector.

6. A valve assembly according to claim 1, wherein:
said outlet is connected to a catheter.

7. A valve assembly according to claim 6, wherein:
said catheter is connected to an inflatable bladder.

8. A valve assembly according to claim 1, wherein:
said second valve assembly is adjustable to open at a selected pressure.

9. A valve assembly according to claim 2, wherein:
said third valve assembly is operable by a push button.

10. A valve assembly for use with a medical infusion system having an inflatable occlusion balloon, an infusion lumen, and an inflatable bladder, said assembly comprising:
a first valve having an inlet and an outlet, said outlet of said first valve being coupled to the balloon;
a second valve having an inlet and an outlet, said outlet of said second valve being coupled to the infusion lumen; and
a third valve having an inlet and an outlet, said outlet of said third valve being coupled to the bladder.

11. A valve assembly according to claim 10, wherein:
said first valve is manually operable,
said second valve operates automatically permitting fluid flow from its inlet to its outlet and preventing fluid flow from its outlet to its inlet, and
said third valve operates automatically permitting fluid flow from its inlet to its outlet and preventing fluid flow from its outlet to its inlet.

12. A valve assembly according to claim 10, further comprising:
a fourth valve located between the inlet and the outlet of said third valve and arranged to automatically open when a preset fluid pressure is exceeded.

13. A valve assembly according to claim 10, further comprising:
a fourth valve assembly located between the inlet and the outlet of said third valve and manually operable to release fluid from the bladder.

14. A valve assembly according to claim 12, wherein:
said fourth valve is adjustable to select the pressure at which it opens.

15. A valve assembly according to claim 12, further comprising:
a fifth valve assembly located between the inlet and the outlet of said third valve and manually operable to release fluid from the bladder.

16. An inflatable balloon for use with a catheter having at least two lumens, said balloon comprising:
an elongate elastic member having an open end and a closed end;
an elastic or superelastic metal wire coupled to the closed and extending at least as far as to the open end,
the open end of the balloon being adapted to couple to an end of the catheter with the wire extending into one of the catheter lumens.

17. A balloon according to claim 16, wherein:
the wire is coupled to the closed end with epoxy.

18. A balloon according to claim 17, wherein:
the closed end is covered with a crimp cap.

19. A catheter and balloon assembly, comprising:
a catheter having at least two lumens;
an balloon having an open end and a closed end, said open end being attached to an end of the catheter;
an elastic or superelastic metal wire coupled to the closed and extending into one of the lumens of the catheter.

20. A catheter and balloon assembly according to claim 19, wherein:
the wire is coupled to the closed end with epoxy and a crimp cap placed over the closed end,
the wire is fixed in the lumen with epoxy, and
the open end is coupled to the end of the catheter with inner and outer crimp bands.

21. An inflatable medical device, comprising:
a catheter having a proximal end, a distal end, and a lumen extending therethrough;
an inflatable member coupled to said distal end;
a valve body having a fluid inlet and a fluid outlet, said fluid outlet being coupled to the proximal end of said catheter;
a first valve assembly located between said inlet and said outlet, said first valve assembly permitting fluid flow from said inlet to said outlet and preventing fluid flow from said outlet to said inlet; and
a second valve assembly arranged to automatically release fluid from said valve body before passing through said outlet when a preset fluid pressure is exceeded.

22. A device according to claim 21, further comprising:
a third valve assembly being operable to allow fluid from said outlet to exit said valve body.

23. A device according to claim 21, wherein:
said second valve assembly is located between said first valve assembly and said inlet.

24. A device according to claim 22, wherein:
said first valve assembly has a movable valve seal, and
said third valve assembly is located between said seal and said outlet.

25. A device according to claim 21, wherein:
said inlet is provided with a Luer connector.

26. A valve assembly according to claim 21, wherein:
said second valve assembly is adjustable to open at a selected pressure.

27. A valve assembly according to claim 22, wherein:
said third valve assembly is operable by a push button.

28. A medical infusion system, comprising:
a catheter having a proximal end, a distal end, and first second and third lumens extending therethrough;
an occlusion balloon coupled to the first lumen;
an infusion device coupled to the second lumen;
an inflatable bladder coupled to the third lumen;
a first valve having an inlet and an outlet, said outlet of said first valve being coupled to the balloon via the first lumen;
a second valve having an inlet and an outlet, said outlet of said second valve being coupled to the infusion device via the second lumen; and
a third valve having an inlet and an outlet, said outlet of said third valve being coupled to the bladder via the third lumen.

29. A system according to claim 28, wherein:
said first valve is manually operable,
said second valve operates automatically permitting fluid flow from its inlet to its outlet and preventing fluid flow from its outlet to its inlet, and
said third valve operates automatically permitting fluid flow from its inlet to its outlet and preventing fluid flow from its outlet to its inlet.

30. A system according to claim 28, further comprising:
a fourth valve located between the inlet and the outlet of said third valve and arranged to automatically open when a preset fluid pressure is exceeded.

31. A system according to claim 28, further comprising:
a fourth valve assembly located between the inlet and the outlet of said third valve and manually operable to release fluid from the bladder.

32. A system according to claim 30, further comprising:
a fifth valve assembly located between the inlet and the outlet of said third valve and manually operable to release fluid from the bladder.
